(19) United States
(12) Patent Application Publication
(10) Pub. No.: US 2004/0110732 A1
(43) Pub. Date: Jun. 10, 2004

(54) Pharmaceutical composition for transdermal or transmucosal administration comprising at least one progestin and/or at least one oestrogen, process for preparing it and uses thereof

(75) Inventors: Valerie Masini-Eteve, Verrieres Le Buisson (FR); Brigitte Taravella, Paris (FR)

Correspondence Address: PIPER RUDNICK, P. O. BOX 64807, CHICAGO, IL, 60664-0807, US

(73) Assignee: BESINS INTERNATIONAL BELGIQUE

(21) Appl. No.: 10/393,077

(22) Filed: Mar. 20, 2003

(30) Foreign Application Priority Data
Dec. 10, 2002 (FR) FR 0215586

(51) Int. Cl. A61K 31/56; A61K 31/57; A61K 31/19

(52) U.S. Cl. 514/170; 514/557

(57) ABSTRACT

The present invention relates to a novel pharmaceutical composition for transdermal or transmucosal administration, comprising

at least one progestin, and/or

at least one oestrogen,

at least one percutaneous absorption promoter which is a hydroxy acid or a pharmaceutically acceptable salt of a hydroxy acid.

The present invention relates to a novel pharmaceutical composition for transdermal or transmucosal administration, comprising at least one progestin and/or at least one oestrogen. The invention also relates to a process for preparing this pharmaceutical composition and to its uses.

In the context of the present invention, the term “progestin” means any steroid having affinities for the progesterone receptors and capable of more or less fully reproducing the biological effects of progesterone.

Progestins thus comprise progesterone and also synthetic progestins. The latter may be classified into three groups (unofficial classification) according to their biological activities (and their structure, which determines said activities); the order of classification thus takes into account their structural difference relative to physiological progesterone.

The first group comprises molecules similar to progesterone or synthetic progestins 1 (SP1) (pregnanes), for example the progesterone isomer (retroprogesterone), Medrogesterone, norprogesterone derivatives (demegestone or promegestone). These molecules have peripheral extra-gestative activity that is virtually identical to that of progesterone, and have no androgenic effects.

The second group comprises 17α-hydroxyprogesterone derivatives or synthetic progestins 2 (SP2) (pregnanes), for example cyproterone acetate and medroxyprogesterone acetate. These molecules have more powerful and more intense peripheral gestative activity than that of progesterone and in addition occasionally have an anti-androgenic effect.

The third group comprises the norsteroids or synthetic progestins 3 (SP3), (estranes or norandrostanes). These are 19-nortestosterone derivatives, for example norethindrone. These molecules have particularly powerful peripheral gestative activity (this is the group of synthetic progestins that has the most pronounced endometrial action) and also have an androgenic effect. From these norandrostanes or estranes are derived molecules of gonane type containing a methyl group at C18 and an ethyl group at C13. Examples that may be mentioned include norgestimate (precursor levonorgestrel), desogestrel (3-keto desogestrel) and gestodene. These chemical changes increase the endometrial power and reduce the intrinsic androgenic activity of the molecule.

A progestin is a compound that is capable, by definition, of maintaining gestation and of promoting the implantation of the egg. This biological role is reflected essentially by a change in the vaginal mucosa (desquamation), in the endometrium (secretory change and predecidualization after oestrogenic impregnation) and in the endocervical glandular epithelium (reduction in the production of glairy mucus and thickening of this mucus).

Progestins also have central action insofar as they regulate the secretion of gonadotrophins via the hypothalamic LH-RH system.

The only physiological effects that all progestin substances have in common are the peripheral effects on the endometrium and the central effects.

The effect on gestation is real for progesterone and very inconsistent with synthetic progestins.

As with progestins, oestrogens also have peripheral physiological effects (proliferative action on the vaginal and uterine mucosi) and central physiological effects. Oestrogens also have pronounced metabolic effects on bone (formation and maintenance of bone mass) and on lipids.

Among the oestrogens, natural, semi-natural and artificial oestrogens are distinguished.

Natural oestrogens, within the strict sense of the term, are represented only by estradiol, more correctly known as 17β-estradiol, the equine conjugated oestrogens, estrone, estriol and phytoestrogens.

Semi-natural oestrogens are derivatives of the above (for example derivatives of ester type), which have in common the ability to be metabolized, at least partially, into natural oestrogens. Among the semi-natural oestrogens that may be mentioned, for example, is estradiol valerate.

Artificial oestrogens are steroids derived from the estrane ring system of 17β-estradiol. The artificial oestrogen most commonly used in oestro-progestin preparations is ethinylestradiol. It has an ethinyl radical in the 17α position on estradiol (17α-ethinyl-17β-estradiol).

During menopause or perimenopause, women suffer an ovarian deficiency which results in addition to loss of reproductive function, in disorders associated with hormonal deficiency and which arise in the short, medium or long term.

Perimenopause is a period of hormonal anarchy, with an occasional return to normal ovarian function. It develops in two stages: the first is characterized by luteal insufficiency and absolute or relative hyperoestrogenism; the second is characterized by the appearance of increasingly long and increasingly frequent periods of hypoestrogenism. The frontiers between these two stages are not clear. The hormonal state and the symptomatology may vary and alternate over time.

Menopause is the period that usually follows perimenopause (it may also occur suddenly without a transition period). It is characterized by the definitive nature of the amenorrhoea and by the absence of secretion of oestrogen.

In physiological terms, perimenopause and menopause correspond, respectively, to a gradual or definitive deprivation of progesterone.

The consequence of the insufficiency of progesterone secretion in women is a loss of its biological effects: progestin effect, anti-androgenic effect (action on the pilosebaceous system and on the skin) and anti-oestrogen effect. This last effect is reflected by hyperoestrogenism. These changes in ovarian activity (of progesterone and of estradiol) may lead to functional impairments and various clinical manifestations, in particular:

premenstrual syndrome,

menstrual irregularities by disovulation or anovulation,

benign mastopathy,

hot flushes,

disorders of the genito-urinary system,

psychogenic difficulties such as anxiety or depression, weight gain, etc.

In therapeutic terms, oestro-progestin replacement treatments have many advantages over regularization of the cycle, disappearance of the discomfort associated with the relative hyperoestrogenism, or even the contraceptive effect.

A pharmaceutical composition based on an oestro-progestin combination has two important actions. The progestin acts to block the ovarian function, which is greatly disrupted in perimenopausal women, and to prevent the development of hyperplasia and cancer of the endometrium associated with oestrogen therapy. The oestrogen makes it possible to limit the disorders typically associated with the menopausal state (hot flushes and other symptoms already mentioned above).

Oestro-progestin formulations for oral administration already exist. However, both for progestins and for oestrogens, the oral route has many drawbacks associated mainly with their high metabolization in the liver (first passage through the liver). Thus, for example, the oral administration of ethinylestradiol, the oestrogen most frequently used in oestro-progestin treatments, is followed by a first passage through the liver, an enzymatic induction factor, the most commonly observed consequences of which being the effects on lipid metabolism (increase in HDL cholesterol, reduction in LDL cholesterol, increase in triglycerides), an increase in the synthesis of angiotensinogen, and a disruption of certain clotting factors. Similarly, the oral administration of oestrogens leads to the appearance of an early and high plasmatic peak with a circulating estradiol/estrone ratio that is very much less than 1, contrary to the physiological situation, and irrespective of the compound administered.

Oral oestro-progestin treatment therefore involves significant risks and side effects. It is to be proscribed especially for the treatment of women having a particular risk factor (changes in the lipoproteinogram, arterial hypertension, diabetes, emboligenic cardiopathy, thromboembolic accidents, etc.)

The harmful effects of an oestro-progestin combination administered orally may be circumvented by the development of formulations for transdermal or transmucosal administration. Specifically, the advantages of percutaneous or transmucosal administration are: in hormonal terms, the production of an estradiol/estrone plasmatic ratio that is closer to the physiological ratio (greater than 1); in metabolic terms, the changes induced by oral oestrogens remain of very low intensity.

“Patches” and other transdermal formulations based on an oestro-progestin combination also exist. In this regard, mention may be made of the following patents and patent applications: U.S. Pat. No. 5,788,984, WO 92/07590, WO 95/17896, WO 97/39743, WO 98/18417, WO 02/11768, EP 0 811 381, and FR 2 814 074.

The Applicant Company has devoted many years of research to the field of hormone therapy, more particularly as regards formulations for transdermal administration. Thus, it has already developed a pharmaceutical composition for transdermal application in the form of an oestradiol-based gel, sold under the brand name Oestrogel®. This pharmaceutical composition has moreover been the subject of a filing, and of a grant of patent FR 2 518 879.

Although transdermal or transmucosal formulations overcome several drawbacks of oral forms (easier application, better patient compliance, elimination of the problem of metabolism by the liver, continuous release over time), they may, however, cause problems with respect to the passage of the active substances through the skin.

In fact, and as specifically explained in international patent application WO 98/18417, a large number of active substances are incompatible with this method of administration since they cannot cross the skin barrier at a speed and a concentration that are sufficient to ensure and maintain a therapeutic plasmatic concentration.

In order to facilitate the passage of the active substances through the skin, the transdermal or transmucosal formulations may include one or more percutaneous absorption promoters.

International patent application WO 92/07590 explains, however, that it is not at all possible to predict the behaviour of an active substance as regards its passage across the transdermal barrier, that this is even more difficult when it involves a combination of active substances, and furthermore that a percutaneous absorption promoter that is effective with respect to a given active substance is not necessarily effective with another active substance.

The Applicant Company continued and completed its research in order to develop an oestro-progestin formulation for transdermal or transmucosal application.

The Applicant Company has thus developed a pharmaceutical composition based on at least one progestin and/or at least one oestrogen, which allows a sufficient passage of the two active substances, or of one of them, across the cutaneous barrier to obtain a therapeutically effective plasmatic level.

The present invention thus relates to a pharmaceutical composition for transdermal or transmucosal administration, comprising:

at least one progestin, and/or

at least one oestrogen,

at least one percutaneous absorption promoter selected from hydroxy acids or pharmaceutically acceptable salts thereof.

The expression “percutaneous absorption promoter” means any molecule that promotes the reversible diffusion of an active principle through the skin or the mucous membranes, and any solubilizing agent that promotes the partition of the active principle between the vehicle and the horny layer of the epidermis or mucous membranes.

Hydroxy acids are widely used in the composition of cosmetic products. They are used essentially in dermatology for treating acne and ageing of the skin (anti-ageing treatment). The mechanism of absorption of hydroxy acids on the skin is still unknown to date. Van Scott et al. have suggested that α-hydroxy acids reduce the cohesion of the cells of the horny layer by modifying the ionic bonds (J. Am. Acad. Dermatol. 1984 11: 867-879).

Hydroxy acids are divided into two groups: α-hydroxy acids such as lactic acid, glycolic acid, malic acid, citric acid, mandelic acid, α-hydroxybutyric acid, α-hydroxyoctanoic acid, pyruvic acid and ethylglycolic acid, and β-hydroxy acids such as salicylic acid.

In the context of the present invention, the terms “hydroxy acid” and “hydroxycarboxylic acid” mean any molecule of the type R—CHOH—COOH comprising inter alia a hydroxycarboxylic function (CHOH—COOH), i.e. a hydroxy alcohol function (CHOH) covalently bonded to a carboxylic function (COOH). The hydroxyacid may comprise several hydroxy functional groups and carboxylic functional groups. The hydroxy acid according to the invention preferably comprises one or two hydroxy groups.

Advantageously, the percutaneous absorption promoter included in the pharmaceutical composition according to the invention is a hydroxy acid. The hydroxy acid is selected for its properties which allow optimal penetration through the skin or the mucous membrane of the active substances present in the pharmaceutical composition according to the invention.

Preferably, the hydroxy acid is either an α-hydroxy acid or a β-hydroxy acid, or a mixture of α-hydroxy acids and/or β-hydroxy acids. Preferably, the hydroxy acid is selected from the group consisting of lactic acid, glycolic acid, malic acid, citric acid, isocitric acid, mandelic acid, benzylic acid, glyceric acid, tartronic acid, α-hydroxybutyric acid, α-hydroxyoctanoic acid, pyruvic acid, ethylglycolic acid, salicylic acid, β-hydroxybutyric acid, aleuritic acid, tropic acid, and mixtures thereof, and even more preferably from the group consisting of lactic acid, glycolic acid, ethylglycolic acid, and mixtures thereof.

A hydroxide is characterised by its pKa. The pKa is the relative strength of the acid and corresponds to its capacity to dissociate into protons (H⁺) in water (Ka=[H⁺]×[A⁻]/[HA]); [H+] is the cation concentration; [A−] is the anion concentration; [HA] is the concentration of non dissociated by hydroxyacid. According to the number of carboxyl functions contained therein, hydroxyacides may have several pKa. α-hydroxyacids, for example, are strong acids and all have weak pKa. For example, mandelic acid has a pKa of 3.41 (at room temperature) and glycolic acid, stronger than mandelic acid, has a pKa of 3.83 (at room temperature).

The hydroxy acid comprised in the pharmaceutical composition according to the invention preferably has all its acid functions in the form of pharmaceutically acceptable salts. The salts will preferably be Li, K, Na, Mg, Ba, Sr, Al, Fe, La, Ce, Mn and/or Zn salts. The salts preferably comprise no heavy metals.

The abovementioned international patent application WO 95/17896 describes monocarboxylic acids containing from 8 to 14 carbon atoms, as percutaneous absorption promoters. It even indicates that the use of monocarboxylic acids containing 7 carbon atoms or less is strongly unadvisable on the grounds that these acids will be too acidic to be administered to the human body.

However, the Applicant Company has found, surprisingly and unexpectedly, that, contrary to what has been taught in the prior art, hydroxy acids, and especially α-hydroxy acids such as lactic acid, glycolic acid, ethylglycolic acid and others, can be very effective as percutaneous absorption promoters in transdermal or transmucosal formulations, without even posing any problems of irritation of the application area.

The pharmaceutical composition according to the invention may also comprise other percutaneous absorption promoters in combination with the hydroxy acids.

Advantageously, the progestin(s) used in the pharmaceutical composition according to the invention may be selected from the group consisting of natural progestins and type 1, 2 or 3 progestins. Preferably, the progestins according to the invention will be of type 3 (SP3) (estranes or norandrostanes), more preferably of gonane type, and even more preferably norgestimate, desogestrel, 3-ketodedogestrel or gestodene.

The oestrogen(s) used in the pharmaceutical composition according to the invention may advantageously be selected from the group consisting of natural oestrogens: 17β-oestradiol, oestrone, equine conjugated oestrogens, estriol and phytoestrogens; semi-natural oestrogens: estradiol valerate; or synthetic oestrogens: ethinyl-estradiol, preferably being 17β-estradiol.

According to one particular embodiment of the pharmaceutical composition according to the invention, the progestin content will be between 0.01% and 5%, preferably between 0.02% and 3% and even more preferably between 0.03% and 1%, these percentages being expressed on a weight basis relative to 100 g of pharmaceutical composition.

According to another particular embodiment of the pharmaceutical composition according to the invention, the oestrogen content will be between 0.01% and 5%, preferably between 0.02% and 3% and even more preferably between 0.03% and 2%, these percentages being expressed on a weight basis relative to 100 g of pharmaceutical composition.

The content of percutaneous absorption promoter(s) in the pharmaceutical composition according to the present invention will advantageously be between 0.1% and 20%, preferably between 0.2% and 10% and even more preferably between 0.5% and 5%, these percentages being expressed on a weight basis relative to 100 g of pharmaceutical composition.

The pharmaceutical composition according to the invention may exist in various forms, for example in the form of a gel, a solution, a cream, a lotion, a spray, an ointment, an aerosol, a patch, a gel capsule or a suppository. The pharmaceutical composition according to the invention is preferably in the form of a gel.

The pharmaceutical composition according to the invention may, in certain cases, also comprise at least one non-aqueous vehicle.

The non-aqueous vehicle must be capable of dissolving the progestin(s) and the oestrogen(s) and also the absorption promoter. It will be chosen from compounds with a low boiling point, i.e. a boiling point of less than 100° C. at atmospheric pressure, such that it can evaporate rapidly on contact with the skin. Such vehicles may be selected from volatile compounds such as ethanol, isopropanol and ethyl acetate; preferably ethanol and/or isopropanol. However, ethanol is a preferred vehicle according to the invention since it contributes efficiently towards the transcutaneous passage of the active principle by evaporating quickly on contact with the skin.

Advantageously, the content of non-aqueous vehicle is between 10% and 90%, preferably between 20% and 80% and even more preferably between 40% and 70%, these percentages being expressed on a weight basis relative to 100 g of pharmaceutical composition.

The pharmaceutical composition according to the invention may also comprise an aqueous vehicle. The aqueous vehicle makes it possible to dissolve the hydrophilic molecules contained in the formulation and also promotes the diffusion of the lipophilic molecules of the formulation towards the horny layer. It may also act as a pH regulator.

The aqueous vehicle may be selected from alkalinizing or basic buffer solutions such as phosphate buffer solution (for example dibasic or monobasic sodium phosphate), citrate buffer solution (for example sodium citrate or potassium citrate), or may simply be purified water. The aqueous vehicle is at a content of between 1% and 80%, preferably between 10% and 70% and even more preferably between 20% and 60%, these percentages being expressed on a weight basis relative to 100 g of pharmaceutical composition.

The pharmaceutical composition according to the invention may also contain a co-solvent such as polyols or polyglycols such as, for example, glycerol (or glycerine), propylene glycol or polyethylene glycol at a content of between 0.5% and 20%, preferably between 3% and 10% and more preferably between 4% and 10%, these percentages being expressed on a weight basis relative to 100 g of pharmaceutical composition. The co-solvent makes it possible to increase the solubility of the active substances.

The pharmaceutical composition according to the invention may, in certain cases, also comprise a gelling agent. Advantageously, and depending on the type of gelling agent used, it has a content of between 0.2% and 30% of a gelling agent, preferably between 0.5% and 10% and even more preferably between 0.3% and 5%, these percentages being expressed on a weight basis per 100 g of pharmaceutical composition.

The gelling agent is preferably selected from the group consisting of carbomers, cellulose derivatives, poloxamers and poloxamines.

Carbomers or polyacrylic acids such as Carbopol 980 or 940 NF, 981 or 941 NF, 1382 or 1382 NF, 5984, 2984 or 934 NF, Pemulen TR1 NF or TR2 NF, Ultrez, Synthalen CR, etc.); cellulose derivatives such as ethylcellulose, hydroxypropylcellulose, hydroxyethylcellulose, hydroxypropylmethylcellulose (HPMC), carboxymethylcellulose (CMC), etc.; poloxamers or polyethylene-polypropylene copolymers such as Lutrol F grade 68 or 127, poloxamines or other gelling agents such as chitosan, dextran, pectins, and natural gums, alone or in combination, may be used in the pharmaceutical composition according to the invention.

These gelling agents make it possible to increase the viscosity of the formulations according to the invention, but may also act as solubilizing agents.

Hydroxypropylcellulose, Carbopol® 980 and Lutrol® are particularly preferred in the context of the present invention.

The gelling agent is selected taking into account the pH of the composition according to the invention and the desired viscosity.

According to another advantageous embodiment of the pharmaceutical composition according to the invention, in the presence of certain types of gelling agents, and in particular non pre-neutralized acrylic polymers, it may contain a neutralizer. The neutralizer/gelling agent ratio is between 10/1 and 0.1/1, preferably between 7/1 and 0.5/1 and even more preferably between 4/1 and 1/1.

This neutralizer is chosen such that it forms, in the presence of the polymer, salts that are soluble in the vehicle.

The neutralizer is also chosen so as to be able to achieve optimum swelling of the polymer chains during the neutralization of the charges and the formation of polymer salts.

According to the invention, triethanolamine is preferably used as neutralizer in the presence of Carbopol® 980. It also allows an optimum viscosity to be achieved in the pharmaceutical composition according to the invention.

Other neutralizers, for instance sodium hydroxide, ammonium hydroxide, potassium hydroxide, arginine, aminomethylpropanol or tromethamine, may be used in the pharmaceutical composition according to the invention. The neutralizer is chosen as a function of the type of gelling agent used, in a manner that is known to those skilled in the art.

Preferably, the pH of the pharmaceutical composition according to the invention will be between 2 and 9, preferably between 3 and 7 and even more preferably between 3 and 6.

The invention also relates to a process for preparing the pharmaceutical composition according to the invention.

This process includes the following successive steps:

progestin(s) and/or oestrogen(s) are dissolved, with stirring, in a mixture of non aqueous vehicle and absorption promoter;

an aqueous vehicle such as water or buffer solution is added, with stirring, to the mixture obtained;

a co-solvent such as propylene glycol is optionally added;

a gelling agent such as hydroxypropylcellulose, Carbopol or Lutrol is then optionally incorporated into the mixture, with stirring;

a neutralizer such as triethanolamine is optionally added to the mixture, with stirring.

The invention also relates to the use of the pharmaceutical composition according to the invention for the preparation of a medicinal product for transdermal or transmucosal application for the treatment of a physiological condition associated with an oestro-progestin deficiency.

Examples of such physiological conditions that may be mentioned include:

disorders of the menstrual cycle or disruptions in the menstrual regularity,

premenstrual syndrome,

mastodynia,

functional ovarian cysts,

mittelschmertz syndrome,

dysmenorrhoea.

The invention will be understood more clearly with the aid of the non-limiting examples described below.

### EXAMPLE 1

### Pharmaceutical Compositions According to the Invention

Gels or solutions according to the invention having the following formulations were prepared by the Applicant Company. The amounts are given per 100 g of pharmaceutical composition:

Formulation A in Gel Form:

| | | |
|---|---|---|
| Gestodene | 0.06 | g |
| 17β oestradiol | 0.12 | g |
| 95% ethanol | 40.00 | g |
| Carbopol 980 NF | 0.50 | g |
| Lutrol F127 | 10.00 | g |
| Lactic acid | 5.00 | g |
| Triethanolamine | 1.50 | g |
| Qs purified water | 100.0 | g |

Formulation B in Gel Form:

| | | |
|---|---|---|
| 3-Ketodesogestrel | 0.06 | g |
| 17β oestradiol | 0.12 | g |
| 95% ethanol | 40.00 | g |
| Hydroxypropylcellulose | 1.50 | g |
| Lactic acid | 5.00 | g |
| Qs purified water | 100.0 | g |

Formulation C in Gel Form:

| | | |
|---|---|---|
| 3-Ketodesogestrel | 0.06 | g |
| 17β oestradiol | 0.12 | g |
| 95% ethanol | 40.00 | g |
| Hydroxypropylcellulose | 1.50 | g |
| Glycolic acid | 5.00 | g |
| Qs purified water | 100.0 | g |

Formulation D in the Form of a Solution:

| | | |
|---|---|---|
| Gestodene | 0.06 | g |
| 17β oestradiol | 0.06 | g |
| 95% ethanol | 40.00 | g |
| Ethylglycolic acid | 5.00 | g |
| Qs purified water | 100.0 | g |

Formulation E in the Form of a Solution:

| | | |
|---|---|---|
| 3-Ketodesogestrel | 0.06 | g |
| 17β oestradiol | 0.06 | g |
| 95% ethanol | 40.00 | g |
| Lactic acid | 5.00 | g |
| Propylene glycol | 5.00 | g |
| Qs purified water | 100.0 | g |

### EXAMPLE 2

### Process for Preparing a Gel According to the Invention

The manufacture of a gel based on estradiol (Diosynth, Netherlands or Schering, Germany) and on 3-ketodesogestrel (Gédéon Richter, Hungary) according to the invention is performed as follows: for a batch of 70 kg containing 0.06% desogestrel and 0.12% estradiol, the process is performed in the following manner:

49 700 g of 95% ethanol are placed in the tank of a Koruma mixer under a vacuum of 800 mbar, with stirring. Next, 42 g of desogestrel are added via the top of the tank. Finally, 84 g of estradiol are added via the top of the tank.

The mixture is mixed for 10 minutes, with the turbomixer at 2 000 rpm and the doctor blade at 40 rpm, until the estradiol and the desogestrel are completely dissolved.

30 674 g of purified water are added under a vacuum of 800 mbar and the mixture is mixed with a doctor blade at 40 rpm.

3 500 g of lactic acid are added via the top and the mixture is mixed for 10 minutes, with the turbomixer at 2 000 rpm and the doctor blade at 40 rpm.

1 050 g of hydroxypropylcellulose (Klucel) (Aqualon, France) are added under a vacuum of 800 mbar. The mixture is mixed at 2 000 rpm. The vacuum is broken. The mixture is mixed for 10 minutes, with the turbomixer at 2 000 rpm and the doctor blade at 40 rpm.

The mixer is placed under a vacuum of 120 mbar for 2 to 3 minutes. Next, the vacuum is broken and the mixture is then stirred for 20 minutes with the doctor blade at 40 rpm.

### EXAMPLE 3

### Process for Preparing a Solution According to the Invention

The manufacture of a solution based on estradiol (Diosynth, Netherlands) and gestodene (Gédéon Richter, Hungary) according to the invention is performed as follows: for a batch of 70 kg containing 0.06% gestodene and 0.12% estradiol, the process is performed in the following manner:

49 700 g of 95% ethanol are placed in the tank of a Koruma mixer under a vacuum of 800 mbar, with stirring. Next, 42 g of gestodene are added via the top of the tank. Finally, 84 g of estradiol are added via the top of the tank.

The mixture is mixed for 10 minutes, with the turbomixer at 2 000 rpm and the doctor blade at 40 rpm, until the estradiol and the gestodene are completely dissolved.

38 374 g of purified water are added under a vacuum of 800 mbar and the mixture is mixed with a doctor blade at 40 rpm.

3 500 g of lactic acid are added via the top and the mixture is mixed for 10 minutes, with the turbomixer at 2 000 rpm and the doctor blade at 40 rpm.

The mixer is placed under a vacuum of 120 mbar for 2 to 3 minutes. Next, the vacuum is broken and the mixture is then stirred for 20 minutes with the doctor blade at 40 rpm.

### EXAMPLE 4

### Tests of In Vitro Percutaneous Absorption of a Transdermal Solution According to the Invention

The percutaneous absorption of ³H estradiol and the effect of various hydroxy acids were studied in Franz-type diffusion cells in vitro.

The in vitro percutaneous absorption was studied quantitatively on biopsies of dermatomed human ventral skin, placed in a 1.77 cm² Franz static diffusion cell, which allows the dermis to be placed in contact with a survival liquid into which the substance absorbed through the skin will be dosed. The survival liquid consists of a solution of 9 g/L sodium chloride supplemented with 15 g/L of serum albumin. The cells are placed under ambient atmosphere and thermostatically maintained at 37° C. 10 μl of preparation are applied to the entire surface of the epidermis circumscribed by the glass cylinder. During the experiment, samples of the survival liquid are taken at times 2 h, 4 h, 6 h, 8 h and 24 h. For each time, the survival liquid taken is replaced with fresh liquid.

Estradiol (Diosynth, Netherlands) was incorporated at 0.06% into aqueous-alcoholic solutions whose absolute ethanol content varied between 40% and 60% (w/w) depending on the solubility of the substances studied.

The studies were performed in the presence of a control corresponding to an aqueous-alcoholic solution of estradiol at 0.06% containing 50% absolute alcohol, by comparison with solutions also comprising lactic acid (Sigma, France), or glycolic acid (Merck, France), or ethylglycolic acid (Sigma, France) in the proportions indicated below.

Results:

Lactic acid at 5% in aqueous-alcoholic solution containing 50% ethanol is capable of significantly increasing the percutaneous absorption of estradiol at 24 hours compared with the control (13.80%±6.78% versus 5.50%±1.76%), and also the flows between 8 and 24 hours. The amounts of estradiol found in the epidermis and the dermis are not changed by the various treatments, and they represent overall between 22% and 26% of the dose applied. This promoting effect of lactic acid depends on this concentration.

The addition of glycolic acid (hydroxyacetic acid) at 5% in an aqueous-alcoholic solution containing 40% absolute ethanol increases the percutaneous absorption compared with the control: 17.73%±2.96% versus 6.96%±2.95% for the control. This effect is not due to the decrease in pH of the formulation resulting from the presence of the glycolic acid: specifically, a control aqueous-alcoholic solution whose aqueous phase was brought to a pH of 2.40 does not lead to a change in absorption. The effects of glycolic acid depend on its concentration in the formulation.

Ethylglycolic acid at 5% also significantly increases the cumulative absorption at 24 hours of estradiol compared with the control (8.90%±1.29% versus 5.26%±1.26%).

1. Pharmaceutical composition for transdermal or transmucosal administration, comprising
at least one progestin, and/or
at least one oestrogen,
at least one percutaneous absorption promoter which is a hydroxy acid or a pharmaceutically acceptable salt of a hydroxy acid.

2. Pharmaceutical composition according to claim 1, in which the hydroxy acid is an α-hydroxy acid preferably selected from the group consisting of lactic acid, glycolic acid, malic acid, citric acid, isocitric acid, mandelic acid, benzylic acid, glyceric acid, tartronic acid, α-hydroxybutyric acid, α-hydroxyoctanoic acid, pyruvic acid, ethylglycolic acid, salicylic acid, β-hydroxybutyric acid, aleuritic acid and tropic acid, and mixtures thereof, and even more preferably from the group consisting of lactic acid, glycolic acid and ethylglycolic acid, and mixtures thereof.

3. Pharmaceutical composition according to claim 1, in which the progestin(s) is(are) selected from the group consisting of natural progestins and progestins of type 1, 2 or 3, preferably type 3 progestins and even more preferably from norgestimate, desogestrel, 3-ketodedogestrel, gestodene and mixtures thereof.

4. Pharmaceutical composition according to claim 1, in which the oestrogen(s) is(are) selected from the group consisting of natural oestrogens: 17β-oestradiol, oestrone, equine conjugated oestrogens, estriol, phytoestrogens; semi-natural oestrogens: oestradiol valerate; or synthetic oestrogens: ethinyl-estradiol, preferably being 17β-estradiol.

5. Pharmaceutical composition according to claim 1, in which the progestin content is between 0.01% and 5%, preferably between 0.02% and 3% and even more preferably between 0.03% and 2%, these percentages being expressed on a weight basis relative to 100 g of pharmaceutical composition.

6. Pharmaceutical composition according to claim 1, in which the oestrogen content is between 0.01% and 5%, preferably between 0.02% and 3% and even more preferably between 0.03% and 2%, these percentages being expressed on a weight basis relative to 100 g of pharmaceutical composition.

7. Pharmaceutical composition according to claim 1, in which the content of percutaneous absorption promoter(s) is between 0.1% and 20%, preferably between 0.2% and 10% and even more preferably between 0.5% and 5%, these percentages being expressed on a weight basis relative to 100 g of pharmaceutical composition.

8. Pharmaceutical composition according to claim 1, which is in the form of a gel, a solution, a cream, a lotion, a spray, an ointment, an aerosol or a patch, preferably in the form of a gel.

9. Pharmaceutical composition according to claim 1, comprising at least one non-aqueous solvent preferably selected from volatile compounds such as ethanol, isopropanol or ethyl acetate, preferably being ethanol and/or isopropanol and even more preferably being ethanol.

10. Pharmaceutical composition according to claim 9, in which the content of non-aqueous solvent is between 10% and 90%, preferably between 20% and 80% and even more preferably between 40% and 70%, these percentages being expressed on a weight basis relative to 100 g of pharmaceutical composition.

11. Pharmaceutical composition according to claim 1, comprising at least one aqueous vehicle selected from the group consisting of alkalinizing or basic buffer solutions such as a phosphate buffer solution such as dibasic or monobasic sodium phosphate, a citrate buffer solution such as sodium citrate or potassium citrate, or purified water.

12. Pharmaceutical composition according to claim 11, in which the content of aqueous vehicle is between 1% and 80%, preferably between 10% and 70% and even more preferably between 20% and 60%, these percentages being expressed on a weight basis relative to 100 g of pharmaceutical composition.

13. Pharmaceutical composition according to claim 1, comprising at least one co-solvent such as polyols or polyglycols such as, for example, glycerol (or glycerine), propylene glycol or polyethylene glycol.

14. Composition according to claim 13, in which the co-solvent content is between 0.5% and 20%, preferably between 3% and 10% and more preferably between 4% and 10%, these percentages being expressed on a weight basis relative to 100 g of pharmaceutical composition.

15. Pharmaceutical composition according to claim 1, comprising at least one gelling agent preferably selected from the group consisting of carbomers, cellulose derivatives, poloxamers and poloxamines.

16. Pharmaceutical composition according to claim 15, in which the content of gelling agent is between 0.2% and 30%, preferably between 0.5% and 10% and even more preferably between 0.3% and 5%, these percentages being expressed on a weight basis relative to 100 g of pharmaceutical composition.

17. Pharmaceutical composition according to claim 15, comprising at least one neutralizer.

18. Pharmaceutical composition according to claim 17, in which the neutralizer/gelling agent ratio is between 10/1 and 0.1/1, preferably between 7/1 and 0.5/1 and even more preferably between 4/1 and 1/1.

19. Pharmaceutical composition according to claim 17, in which the neutralizer(s) is(are) selected from the group consisting of triethanolamine, sodium hydroxide, ammonium hydroxide, potassium hydroxide, arginine, aminomethyl propanol and tromethamine.

20. Pharmaceutical composition according to claim 1, having a pH of between 2 and 9, preferably between 3 and 7 and even more preferably between 3 and 6.

21. Process for preparing the pharmaceutical composition in the form of a gel according to claim 1, comprising the following successive steps:
progestin(s) and/or oestrogen(s) are dissolved, with stirring, in a mixture of non aqueous vehicle and absorption promoter;
an aqueous vehicle such as water or buffer solution is added, with stirring, to the mixture obtained;
a co-solvent such as propylene glycol is optionally added;
a gelling agent is then incorporated into the mixture, with stirring;
a neutralizer is optionally added to the mixture, with stirring.

22. Method for the treatment of a physiological condition associated with an oestro-progestin deficiency, comprising administering an effective amount of a pharmaceutical composition according to claim 1 to a subject.

23. Method according to claim 22, in which the physiological condition is selected from the group consisting of:
disorders of the cycle or disruptions in the menstrual regularity,
premenstrual syndrome,
mastodynia,
functional ovarian cysts,
mittelschmertz syndrome,
dysmenorrhoea.
